(19) United States
(12) Patent Application Publication
(10) Pub. No.: US 2022/0183750 A1
(43) Pub. Date: Jun. 16, 2022

(54) Systems for Treating Arrhythmia by Pulsed Field Ablation

(71) Applicant: SHANGHAI OPTIPULSE BIOTECH CO.,LTD, Shanghai (CN)

(72) Inventors: Longteng BAI, Beijing (CN); Jiahong TAN, Shanghai (CN)

(21) Appl. No.: 17/653,283

(22) Filed: Mar. 2, 2022

Related U.S. Application Data
(63) Continuation of application No. PCT/CN2021/091680, filed on Apr. 30, 2021.

(30) Foreign Application Priority Data
Jul. 6, 2020 (CN) 202010638621.8
Jul. 6, 2020 (CN) 202021292858.7

(51) Int. Cl. A61B 18/14; A61B 18/12

(52) U.S. Cl. CPC A61B 18/1492; A61B 2018/00577; A61B 18/1206

(57) ABSTRACT

The purpose of this invention is to provide pulsed field ablation (PFA)-based systems and devices for treating arrhythmia, which includes a pulsed voltage console, a pacing and ECG unit, and an ablation catheter. The pulsed voltage console is comprised of an electric pulse generator, a controller, a user interface(UI), and a converter. The pacing and ECG unit is comprised of an ECG recorder, a pacing catheter, a cardiac stimulator, and a mapping catheter. The pacing electric signal is transmitted to the pulsed voltage console. Voltage pulse delivery is synchronized to join pacing signal. Based on the pacing signal, a voltage pulse waveform is delivered during the refractory period of the cardiac cycle. The ablation catheter is connected to the system console through a converter, transferring the electric field energy to the tissue through electrodes on the ablation catheter. The ablation catheter includes a spline basket. The distal end of the spline basket is coupled with an annular catheter that can go into pulmonary vein (PV). Irreversible electroporation can be formed locally, linearly, or circularly, and evenly distributed over a large area, achieving the purpose of effectively treating atrial flutter, supraventricular tachycardia, atrial fibrillation, and other arrhythmias.

### CROSS-REFERENCE TO RELATED APPLICATION

This application is a continuation of International Patent Application Number PCT/CN2021/091680, filed on Apr. 30, 2021, which claims the benefit and priority of Chinese Patent Application Number, CN202010638621.8 and CN202021292858.7, filed on Jul. 6, 2020. The entire disclosure of each of the foregoing applications is incorporated herein by reference in its entirety.

### FIELD OF THE INVENTION

The present disclosure belongs to the field of medical devices, and relates to medical systems and methods of use thereof for treating cardiac tissue using pulsed field ablation (PFA) technology, in particular towards treating arrhythmia using multipolar catheter-based irreversible electroporation (IRE) ablation.

### BACKGROUND OF THE INVENTION

Since the first implementation of cardiac ablation in 1969, there have been numerous innovations and rapid developments in the field. Ablation was first used in treating supraventricular tachycardia with auxiliary pathways and pre-excitation syndrome. Today, ablation is applied in the treatment for atrial flutter, atrial fibrillation (AF), and ventricular arrhythmia.

The purpose of ablation is to destroy potential arrhythmia tissues, and form transmural and continuous permanent lesion. Percutaneous catheter ablation, which employs radio-frequency ablation (“RFA”) or cryoablation to achieve the pulmonary vein (PV) isolation in atrial tissue, has become a widely accepted interventional method for treating AF. Other energy forms developed for catheter-based ablation include microwaves, high-intensity focused ultrasound, low-intensity collimated ultrasound, lasers, cryogenic energy, and heated saline.

Currently, radiofrequency (RF) energy is the most commonly used energy source. RF induces lesion through heating the surface tissue with electric resistance and transferring the heat to deeper tissues. It is quite effective; however, due to thermal conductivity, it causes adverse effects not only to the target tissue but also to the surrounding tissues. Especially in the process of radiofrequency ablation, heat transfer can cause esophageal injury (forming esophageal fistula), septal nerve damage, pulmonary vein stenosis, coagulum/thrombosis, and subsequent risk of thromboembolism, which may all cause cerebral infarction or injury.

Apart from RF, cryoablation is another widely used method. Cryoablation freezes and destroys tissue with extreme cold. However, similar to RF, cryoablation also induces complications that include esophageal fistula, PV stenosis, nerve paralysis, and potential pulmonary hemoptysis. Although these two energy sources for ablation are effective to a large extent, alternative energy sources are still used in the hope to improve ablation safety.

Irreversible electroporation (IRE) is a rapidly developing, well-recognized, and FDA-approved treatment for solid tumors. It was recently approved for treating pancreatic cancer. Pulsed direct current (DC) can be used to generate a local electric field to affect the permeability of a cell membrane's lipid bilayer, thereby inducing the formation of nano-scale defects or pores to increase permeability. Depending on the parameters of the electric pulse used (e.g., pulse duration, voltage, frequency), this may be a reversible process, in which the cells survive the restoration via cell membrane integrity and homeostasis, or an irreversible process, in which the electroporation leads to cell death.

IRE may be a promising approach for cardiac ablation. Especially when compared with RF, IRE can produce ablation-induced lesions without the consequences of heat conduction, that is, it can preserve the surrounding tissue structure. In this field, IRE is more commonly referred to as Pulsed Field Ablation (PFA). Due to the potential advantages of PFA over current ablation methods, there have been many preclinical studies with animals; and recently, data on the first short-term clinical research with humans is published. PFA treatment for acute isolation of PV using circular PV ablation catheter to deliver pulsed electric fields to produce myocardial infraction, has a success rate of 92.0%. The result demonstrates that this method is a new, potential, quick, and safe ablation method. Reddy et al. concluded from the results of two small, short-term human clinical studies, that by improving PFA ablation parameters, the success rate of PV isolation in 3 months was 100% with no observation of stroke, nerve damage, PV stenosis, and esophageal injury. The success rate of 12 months free of arrhythmia was 87.4%.

The systems of this patent are designed for the innovations surrounding PFA-based ablation, that can significantly improve ablation efficiency and safety, be applied to the field of arrhythmia treatment, and hopefully achieve the purpose of a rapid, safe and effective treatment for arrhythmia and other diseases.

### SUMMARY OF THE INVENTION

The purpose of this invention is to provide PFA-based systems for treating arrhythmia, which includes a pulsed voltage console, a pacing and ECG unit, and an ablation catheter.

The pulsed voltage console is comprised of an electric pulse generator, a controller, a user interface(UI), and a converter.

The pacing and ECG unit is comprised of an ECG recorder, a pacing catheter, a cardiac stimulator, and a mapping catheter. The pacing electric signal is synchronously transmitted to the pulsed voltage console.

The ablation catheter is comprised of control handle of the distal, middle, and proximal sections connected in sequence.

The ablation catheter is connected to the system console through a converter, based on the pacing signal, delivering a voltage pulse waveform during the refractory period of the cardiac cycle, transferring the electric field energy generated from the pulse generator to the tissue to be ablated through the electrodes on the ablation catheter. During the ablation discharge, the converter isolates the pacing and ECG unit from the pulse system console.

The distal section of the ablation catheter is comprised of a spline basket, which is formed by at least one soft, flexible spline with at least one electrode attached on its surface.

In the spline basket structure, each spline including 2 to 4 electrodes.

Each spline basket is preferably made up of either 1 or 4-10 splines. In one embodiment, the spline basket is consisted of 1 spline. In another embodiment, the spline basket is consisted of 4 to 10 splines.

In one embodiment, the tubing of the spline's main body is made from a flexible, insulating polymeric materials. The insulated wire is connected to the electrode embedded on the surface of the spline, and attaches to the electric socket on the control handle through the main body of the catheter.

In a preferred embodiment, the tubing of the spline has a dimension of an outer diameter of 0.2-3 mm, an inner diameter of 0.1-2.9 mm, and a length of 10-60 mm.

In another embodiment, the proximal end of the soft, flexible spline is connected to the middle section of the body of the catheter. The distal end of the spline is fixed on a guide shaft with an inner lumen, the guide shaft is directly connected to the knob or push button of the control handle in the proximal portion of the catheter; the guide shaft can also be connected to the handle by a pull wire; by controlling the handle remotely, the splines in the distal section of the catheter can be formed into a spline basket or retracted into its extended state.

Splines can be evenly distributed on a 360-degree basket-shaped sphere in a three-dimensional space when multiple splines exist and form a basket.

In some embodiments, each electrode on the spline is annular shaped, with an outer diameter of 0.3-3 mm and a length of 1-20 mm; the electrodes are insulated and separated by elastic-insulating polymeric materials with an electric insulation of above 500V.

In some embodiments, the pulsed voltage console can trace each electrode on the spline. This means that electrodes on adjacent splines can be selected for paired positive and negative discharges, or different electrodes on the same spine can be selected to perform paired positive and negative discharge to conduct ablation.

In some embodiments, an annular catheter is connected to the distal end of the spline basket of ablation catheter. The structure of this annular catheter is preferably in the formation of either circular with one ring, or cylindrical or conically helical with more than two rings. There must be at least one electrode on the annular catheter.

Preferably, the annular catheter has an outer diameter of 10-30 mm in its extended state, and 5-15 electrodes with a length of 1-4 mm.

Further, the pulsed voltage console can trace each electrode on the circular catheter to select the electrode therein to discharge for ablation or pair the electrode with those on the spline basket for paired discharge ablation.

In some embodiments, two adjacent electrodes on the annular catheter are set to be one positive and one negative to conduct PFA sequentially or simultaneously.

The technical benefits obtained in this invention:

1) The ablation catheter includes a spline basket and the distal end of the spline basket is coupled with an annular catheter that can go into the PV. In addition to pairing the electrodes on the spline basket contacting with the PV orifice for ablation, the electrodes on the circular catheter can also be paired for discharge ablation inside the PV. Furthermore, electrodes on the spline basket and those on the annular electrodes can be paired for bipolar discharge ablation. This design can enlarge the range of ablation from the traditionally circular ablation around the PV orifice to areas inside the PV and the cylindrical gap between the two rings. This enables a rapid expansion of the ablating area and can achieve the purpose of longer-lasting, effective PV isolation.

2) By selectively controlling the electrodes on the spline basket and the annular catheter to discharge ablation, irreversible lesions can be formed locally, linearly, circularly, or evenly distributed over a large area, achieving the purpose of effectively treating atrial flutter, supraventricular tachycardia, atrial fibrillation, and other arrhythmias.

3) The annular catheter can enter PV through the inner lumen of ablation catheter (called guide wire lumen) and better fix the spline basket onto the PV orifice using the annular catheter for positioning inside PV, improving the electrode connectivity with the tissue and ablation efficiency at the PV orifice, achieving complete PV isolation. In addition, the annular catheter can also detect the effectiveness of PV isolation in a much timely manner.

### BRIEF DESCRIPTION OF THE DRAWINGS

The drawings described here are used to provide a more comprehensive understanding of the present invention and constitute as part of this application. The exemplary experiments of the present invention and their descriptions are used to for explanatory purpose, and do not constitute as improper limitations of the present invention. The attached drawings include:

FIG. 1 is an illustration of the structure of the PFA system of the present invention;

FIG. 2 is an illustration of the overall structure of the PFA catheter of the present invention;

FIG. 3 is an illustration of the structure of the spline basket in one experiment of the present invention;

FIG. 4 is an illustration of the structure of the spline basket in a second experiment of the present invention;

FIG. 5 is an illustration of the structure of the spline basket in a third experiment of the present invention;

FIG. 6 is an illustration of the structure of the spline basket in a fourth experiment of the present invention;

FIG. 7 is an illustration of the structure of the annular catheter in one experiment of the present invention;

FIG. 8 is an illustration of the structure of the annular catheter in a second experiment of the present invention;

FIG. 9 is an illustration of the structure of the annular catheter in a third experiment of the present invention;

FIG. 10 is an illustration of the overall structure of the distal catheter in one experiment of the present invention;

FIG. 11 is an illustration of the structure of the distal catheter in its extended form in one experiment of the present invention.

### DETAILED DESCRIPTION OF THE INVENTION

Hereinafter, the detailed technical method of the present invention will be described through experiments with reference to the accompanying drawings. It should be noted here that the description of these experiments is used to help understanding the present invention, but does not constitute as limitation to the present invention.

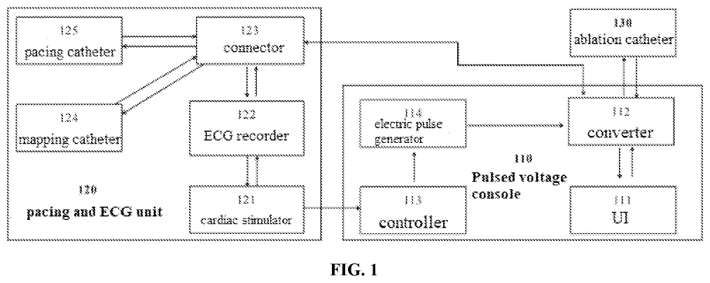

As shown in FIG. 1, it is a system for arrhythmia treatment using PFA technology, which mainly includes a pulsed voltage console 110, a pacing and ECG unit 120, and an ablation catheter 130.

The pulsed voltage console 110 includes an electric pulse generator 114, a controller 113 (embedded with processor), a display-included user interface 111 (UI) , and a converter 112.

The ablation catheter 130 connects to the system console through the converter 112, transmitting the pulsed electric field to the tissue to be ablated through electrodes on the ablation catheter; during the ablation discharge, the converter isolates the pacing and ECG unit from the pulsed voltage console.

The pacing and ECG unit 120 includes a cardiac stimulator 121, an ECG recorder 122, a mapping catheter 124, a pacing catheter 125, and a connector 123. The pacing electric signal is transmitted synchronously to the pulsed voltage console 110. Based on the pacing signal, within the refractory window, the system console 110 sends out ablating pulses to the tissue. In the experiment, the refractory window follows the ventricular pacing signal immediately, or with a very short lag, and lasts no more than 130 ms, where the entire ablation discharge is within this time interval.

The ablation catheter 130 includes, in the sequence of connection, a distal section 131 (within the body), a middle section 132, and a proximal section 133.

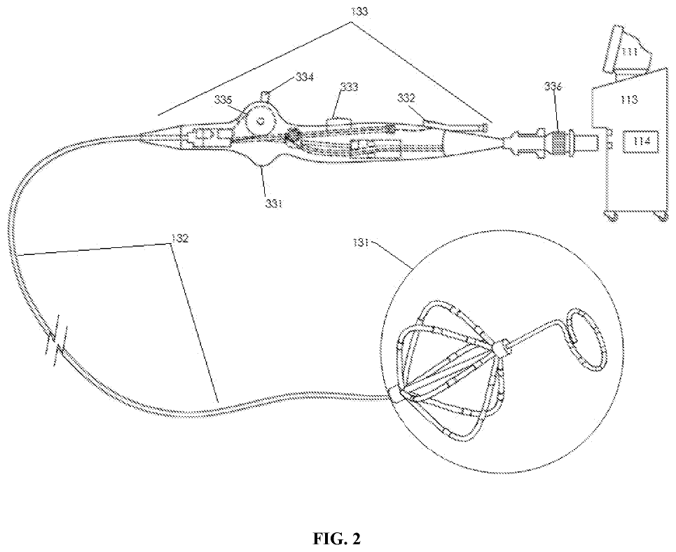

As shown in FIG. 2, FIG. 2 is a schematic diagram of the overall structure of the PFA catheter of the present invention. Wherein, the distal section 131 is comprised of a treatment head, such as the spline basket and/or the annular catheter.

The middle section 132, the main body, is an elongated catheter that has a hollow inner lumen where electric catheter, wires, guide wire, and etc. are arranged within.

The proximal section 133 is composed of a control handle 331, which includes receiving wires or components of other medical treatment devices 332, and a connector 336 to the main body of the handle. The handle 331 may include a wire-drawing assembly 335 used to manipulate the treatment head of the distal section 131, a lever or knob 334, and an actuator 333. The proximal end of the wire-drawing assembly 335 may be anchored to component that can communicate with and respond to the lever or knob 334, such as a cam. The actuator 333 is coupled to the proximal section of the catheter and/or the handle 331 in a linkable manner to manipulate and move the treatment head on the distal section 131. The actuator may include a sliding key, a button, a rotating rod, or other mechanical structure attached to the handle or the catheter in a linkable fashion.

The main body 132 is a braided mesh tube with excellent torsion control. The inner lumen of the braided tube can be a single- or multi-cavity structure made from insulating material such as TPU or PEBAX, or from polymeric materials with small friction coefficient and good insulation properties such as polyimide, FEP, ETFE, or PTFE. The middle braided mesh comprises of stainless steel, Nitinol, and other alloy coils. The outer layer is made up with biocompatible electric-insulating materials such as TPU, PEBAX, nylon, and other materials.

The single lumen liner of the braided middle section of the ablation catheter 132, is made from TPU, PEBAX, silicone rubber, polyimide, FEP, ETFE, or PTFE tube to form the guide wire cavity extending into the spline basket of the distal section. The proximal section enters the handle and forms the guide wire cavity when joined with the Luer connector. The guide wire and the annular mapping catheter pass through the cavity to reach the PV directly.

The distal section 131 of the ablation catheter may also be a balloon covered by mesh, utilizing electrodes embedded on the surface of the balloon to perform discharge ablation.

The distal portion 131 of the ablation catheter may also have an annularly multi-polar structure fitted to the PV orifice with an outer diameter of 2 to 5 cm and an electrode count of 4 to 16. The two adjacent electrodes are set to be opposing anode and cathode, completing a pulse discharge ablation in a sequential manner to perform a full PV isolation.

The ablation catheter is connected to the system console through a converter. Based on the pacing signal, this pulse generator is programmed to deliver a high-voltage pulse to the electrode during the refractory period, sufficient to cause irreversible electroporation of myocardial tissue cells. The pulse may be unidirectional, bidirectional, or other combinations. The voltage ranges from 100 to 3500 volts with pulse widths in the range of 10 to 1500 microseconds, pulse intervals in the range of 10 to 2000 microseconds, and pulse sequences in the range of 1-500 milliseconds. Each ablation site can be a single-pulsed ablation or a multi-pulsed ablation that cause irreversible electroporated lesions of the tissue.

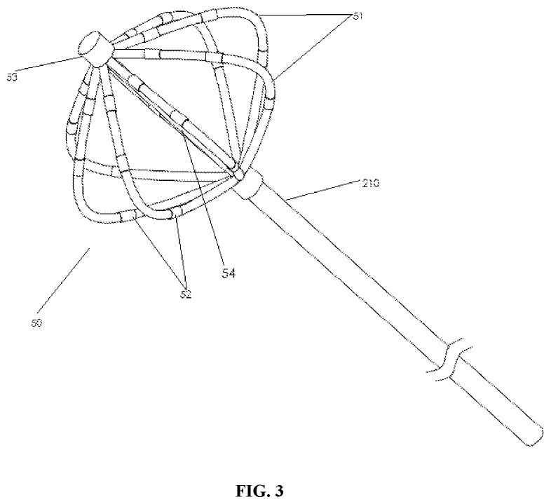

As shown in FIGS. 3-6, in the treatment head component attached to the distal section 131, the spline basket 50 having a basket-like shape is preferred to have 1 or 4-10 multiple soft, flexible splines.

Each spline contains at least one electrode 52 which is responsible for transmitting high-voltage pulses to the tissue and mapping. The spline basket includes 2 to 14 soft, flexible splines 51, preferably 4 to 10 splines 51. Each spline 51 has 1 to 6 conductive electrodes 52, preferably 2 to 4 electrodes 52.

The spline basket is consisted of one or more splines 51 made of flexible, insulating polymeric materials. The insulated wire inside insulating soft polymer tubing is connected to multiple electrodes 52 embedded on the surface of the spline, and the insulating wire passes through the main body of the catheter to the electric socket of the control handle. The spline is composed from flexible insulating polymeric materials, including but not limited to polyimide, FEP, TPU, PEBAX, nylon, and silicone. The insulating wire within the insulated polymer catheter is connected to the electrodes embedded on the surface of the spline, where the insulated wire connects to the electric socket on the distal handle via main body of the catheter.

Preferably, the annular tubing of the spline 51 is to have an outer diameter of 0.2-3 mm, an inner diameter of 0.1-2.9 mm, and a length of 10-60 mm.

In some embodiments, the proximal end of the soft, flexible spline basket 50 is connected to the middle section of the catheter 210; the distal section of the spline is fixed to the cavitated fixture 53. The component and the cavitated guide shaft 54 are connected to the rotary handle or the push lever on the proximal control handle through a pull wire. The distal splines can be formed into a spline basket or retracted into its extended state by controlling the handle.

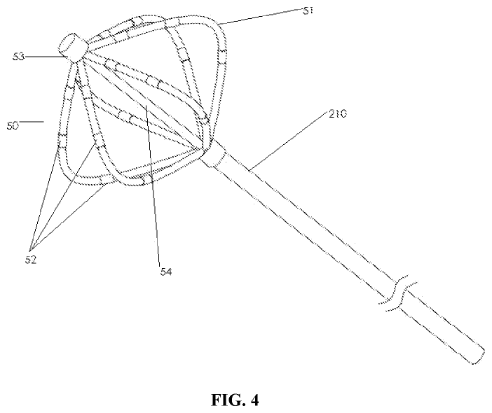

As shown in FIG. 3, In some embodiments, the spline basket 50 includes 8 splines 51. As shown in FIG. 4, In some embodiments, the spline basket 50 includes 6 splines 51.

When there are multiple splines 51, the spline basket 50 forms a basket shape with splines evenly distributed on a 360-degree basket-shaped sphere in the three-dimensional space.

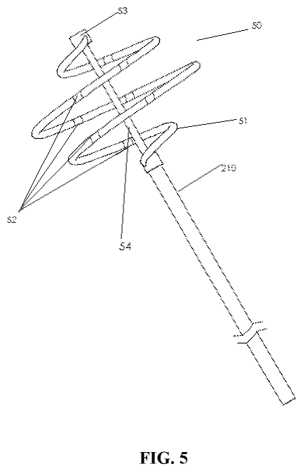

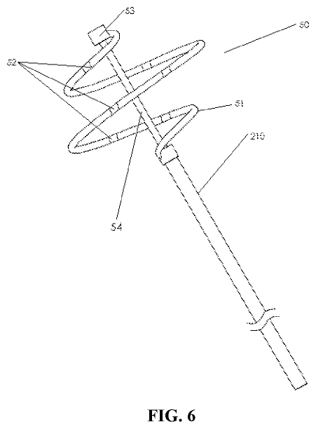

As shown in FIGS. 5 and 6, In some embodiments, the spline basket 50 composes an annular tubing 51 that forms a helical basket-like structure, with a wider middle portion and two narrow ends.

In some embodiments, every electrode 52 on the spline is annularly shaped with an outer diameter of 0.3-3 mm and a length of 1-20 mm. Multiple electrodes 52 are separated and isolated by electrical-insulating polymeric materials with an electric insulation of above 500V.

In some embodiments, the pulsed voltage console can trace every electrode 52 on the spline to select the electrode 52 on adjacent splines for positively and negatively paired discharge. Different electrodes 52 on the same spline can also be paired to perform positive and negative discharge ablation.

As shown in FIGS. 3-6, the proximal end of multiple soft, flexible splines is connected to the catheter 210 in the middle section of the catheter body, with each spline's distal section fixated on the fixture with inner cavity 53.

In an experiment, the ablation catheter handle 331 is composed of a mechanism with a sliding rod, a gear, and a pull wire. The pull wire of one of the mechanisms is connected to the spline basket 50 through rotation or push-and-pull of the handle 331, forming the spline basket or straightening the spline to retract the spline basket for preparing of the reposition or the ablation of other PVs. The pull wire of another control mechanism is connected to the proximal end of the spline basket and controls the directions of the spline basket by a rotatory knob or a push button, fitting the spline basket to the PV orifice seamlessly from various directions.

The fixture 53 is connected to the rotary handle or push rod of the proximal handle through a pull wire, forming the splines into spline basket or retracting the spline basket into its straightened state in the distal section using the handle. In the spline basket state, splines evenly distributed on the 360-degree basket-shaped sphere in the three-dimensional space.

Every electrode on the soft, flexible spline is annularly shaped with an outer diameter of 0.3-3 mm, a length of 1-20 mm, and made with selective materials such as platinum, platinum alloy, gold, gold alloy, silver, stainless steel, nickel titanium alloy, and graphene. The electrodes are separated and isolated by electric-insulating polymeric materials with an electric insulation above 500V.

The pulsed voltage console 110 can trace every electrode 52, select electrodes 52 on adjacent splines for positively and negatively paired discharge. Different electrodes 52 on the same spline can also be paired perform positive and negative discharge ablation. Other combinations are, too, permitted.

The guide wire lumen in the center of the spline basket is composed of insulating materials such as polyimide, PEEK, PTFE, FEP, ETFE, TPU, and PEBAX.

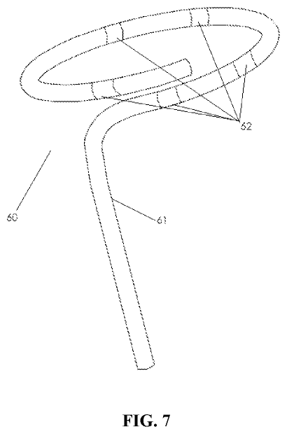

As shown in FIGS. 7-9, the ablation catheter has another configuration: besides the spline basket in the distal section, there is an annular catheter 60, composed of an insulated tubing 61, that is attached to the distal section of the spline basket. The outer surface of this annular catheter 60 consists of multiple electrodes 62.

The annular tubing 61 is made from soft, insulating polymeric materials, including but not limited to polyimide, FEP, TPU, PEBAX, nylon, and silicone. The insulting wire within the soft insulated polymeric tubing is connected to electrodes embedded on the surface of the spline and is attached to the electrical socket at the proximal section of the handle through the main body of the catheter.

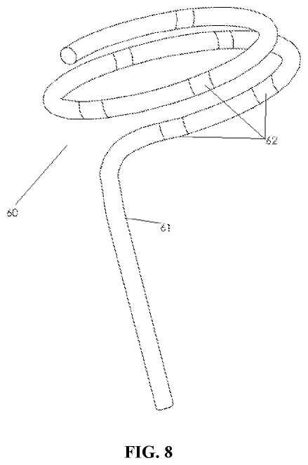

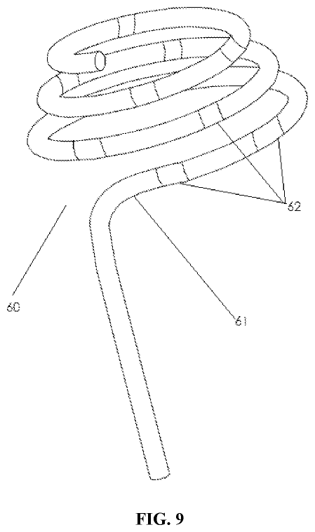

As shown in FIGS. 7-9, the preferred configuration for the annular catheter includes a annularly formed ring (FIG. 7), two or more annularly formed cylinder (FIG. 8), or helical cone (FIG. 9).

In some embodiments, the distal annular catheter 60, under its extended state, has an outer diameter of 10-30 mm, preferably 15-20 mm; an electrode count of 5-15, preferably 6-10; and a length of 1 to 4 mm, preferably 1.5 to 3 mm.

This annular catheter 60 can enter the PV to effectively detect the PV isolation and can also discharge ablation. The annular catheter enters the PV through the guide wire lumen of the ablation catheter.

In some embodiments, the two adjacent electrodes 62 in the annular catheter 60 are set as positive and negative electrodes, performing discharge ablation sequentially or simultaneously to form a complete PV isolation.

Further, the pulsed voltage console 110 can trace every electrode 62 on the annular catheter to select any electrode 62 therein for discharge ablation or pair with the electrode on the spline basket for combined discharge ablation.

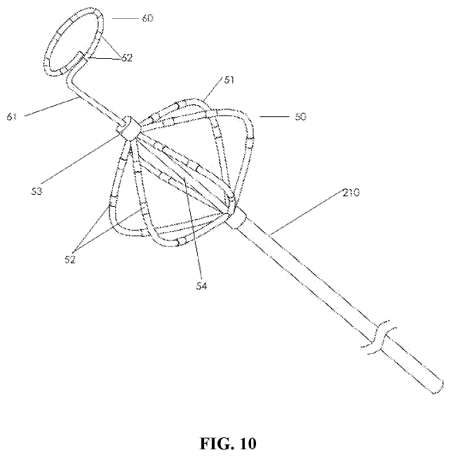

As shown in FIG. 10, the main annular wire 61 of the annular catheter 60 extends from the inner lumen of the guide shaft 54 of the spline basket 50 through the inner cavity of the fixture 53. Among them, multiple proximal sections of the soft, flexible splines 51 are connected to the middle section of the catheter 210. The distal section of every spline 51 from the spline basket 50 is anchored onto the cavitated fixture 53 and the guide shaft 54 can extend and contract through the catheter 210, enabling the control of the expansion of the spline basket. The proximal control handle can control the extension of the annular catheter 60 through the guide wire.

In some embodiments, the pulsed voltage console 110 can trace every electrode 62 of the annular catheter and every electrode 52 of the spline basket, and select adjacent electrode pair 62 to perform discharge ablation, thereby achieving a three-dimensional cylindrical ablation.

Variable combinations of electrode arrange on the tissue-contacting distal section of the catheters and the different traceable electrodes can form different high-voltage pulsed electric fields. For example, by adjusting the electrode location and electrode potential, the electrodes on the annular catheter and those on the spline basket can discharge on a multiple combination basis, enlarging the discharge area, which results in a larger and more sufficient discharge ablation area compared to that from two adjacent electrodes solely. In turn, the combined discharge ablation can form irreversible lesions locally, linearly, circularly, helically, or evenly spread over a large area, so as to achieve the purpose of long-term treatment for atrial flutter, supraventricular tachycardia, atrial fibrillation, and other cases of arrhythmia.

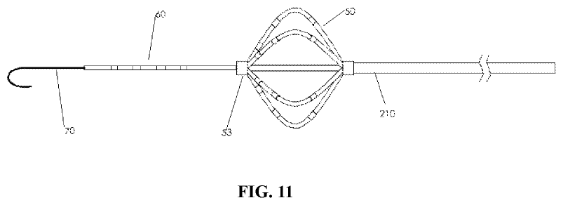

As shown in FIG. 11, it is a schematic diagram of the structure of the extended distal annular catheter during an experiment of this invention. Wherein, the cavity-embedded guide wire 70 extends out of the annular catheter 60. The annular catheter 60 can be extended into a linear shape to facilitate movement inside the blood vessel. By withdrawing the guide wire, the annular catheter returns to its soft, flexible annular form, automatically adapting to the size of the PV.

Only the preferable experiments are selectively included and are not limited to the particulars of the present invention herein above. To the skilled persons in this art, various modifications and amendments can be made to the present invention. Any edit, equivalent replacement, improvement, and etc. made within the spirit and principles of invention shall be protected within the scope on the present invention.

We claim:

1. A medical system for treating arrhythmia by pulsed field ablation, comprising: a pulsed voltage console, a pacing and ECG unit, and an ablation catheter; wherein
the pulsed voltage console is comprised of an electric pulse generator, a controller, a user interface, and a converter;
the pacing and ECG unit is comprised of an ECG recorder, a pacing catheter, a cardiac stimulator, and a mapping catheter. The pacing electric signal is synchronously transmitted to the pulsed voltage console;
the ablation catheter is comprised of the distal, middle, and proximal (control handle) sections connected in sequence;
the ablation catheter is connected to the console through a converter, based on the pacing signal, delivering a voltage pulse waveform during the refractory period of the cardiac cycle, transferring the electric field energy generated from the pulse generator to the tissue to be ablated through the electrodes on the ablation catheter. During the ablation discharge, the converter isolates the pacing and ECG unit from the pulse system console.

2. The medical system of claim 1, wherein the distal section of the ablation catheter is comprised of a spline basket, which is formed by at least one soft, flexible spline with at least one electrode attached on its surface.

3. The medical system of claim 2, wherein the spline basket is preferably made up of either 1 or 4-10 splines, each spline including 2 to 4 electrodes.

4. The medical system of claim 3, wherein the tubing of the spline's main body is made from a flexible, insulating polymeric materials; the insulating wire inside the insulated polymeric tubing is connected to the electrode embedded on the surface of the spline, and attaches to the electric pin of socket inside the control handle through the main body of the catheter.

5. The medical system of claim 4, wherein the tubing of the spline has a dimension of an outer diameter of 0.2-3 mm, an inner diameter of 0.1-2.9 mm, and a length of 10-60 mm.

6. The medical system of claim 4, wherein the proximal end of the spline is connected to the middle section of the catheter body;
the distal end of the spline is fixed on a guiding shaft with an inner lumen;
the guiding shaft is directly connected to the knob or push rod of the control handle in the proximal portion of the catheter; the guiding shaft can also be connected to the handle by a pull wire;
by controlling the handle, the splines in the distal section of the catheter can be formed into a spline basket or retracted into its extended state.

7. The medical system of claim 2, wherein each electrode on the spline is annular shaped, with an outer diameter of 0.3-3 mm and a length of 1-20 mm; the electrodes are insulated and separated by elastic-insulating polymeric materials with an electric insulation of above 500V.

8. The medical system of claim 3, wherein the pulsed voltage console can trace each electrode on the spline;
the electrodes on adjacent splines can be selected for paired positive and negative discharges, or different electrodes on the same spine can be selected for paired positive and negative discharge to perform ablation.

9. The medical system of claim 2, wherein an annular catheter with at least one electrode attached on its surface is coupled to the distal spline basket of the ablation catheter.
The structure of this annular catheter is preferably in the formation of either circular with one ring, or cylindrical or conically helical with more than two rings, at least one electrode is embedded on the surface of the annular catheter

10. The medical system of claim 9, wherein the annular catheter has an outer diameter of between 10 and 30 mm in its extended state, and 5-15 electrodes with a length of 1-4 mm.

11. The medical system of claim 9, wherein the pulsed voltage console can trace each electrode on the circular catheter to select the electrodes therein to discharge for ablation or pair the electrodes with those on the spline basket to perform ablation.

12. The medical system of claim 9, wherein two adjacent electrodes on the annular catheter are set to be one positive and one negative to conduct PFA sequentially or simultaneously.
